(19) United States
(12) Patent Application Publication
(10) Pub. No.: US 2008/0268043 A1
(43) Pub. Date: Oct. 30, 2008

(54) Controlled Release Compositions Comprising an Antipsychotic Agent

(75) Inventors: Scott A Jenkins, Downingtown, PA (US); Gary Liversidge, Westchester, PA (US)

Correspondence Address: Fox Rothschild, LLP;Elan Pharma International Limited, 2000 Market Street, Philadelphia, PA, 19103, US

(73) Assignee: ELAN PHARMA INTERNATIONAL LIMITED, Monksland Athlone, County Westmeath (IE)

(21) Appl. No.: 11/569,483

(22) PCT Filed: Jan. 26, 2006
(86) PCT No.: PCT/US2006/002751

§ 371 Date: Feb. 1, 2008

Related U.S. Application Data
(60) Provisional application No. 60/647,311, filed on Jan. 26, 2005.

(51) Int. Cl. A61K 9/52; A61K 31/5415; A61K 33/00; A61K 9/14; A61K 31/19; A61K 31/554

(52) U.S. Cl. 424/458; 514/224.8; 424/722; 514/557; 514/211.13; 424/490

(57) ABSTRACT

The invention relates to a formulation for the treatment of acute manic episodes associated with Bipolar I Disorder comprising an antipsychotic agent selected from the group consisting of: a dibenzothiazepine derivative; lithium; and divalproex. The formulation comprises a first component which comprises a first population the antipsychotic agent and at least one subsequent component which comprises a subsequent population of the antipsychotic agent and which allows for the modified release of the agent. The combination of the first and the subsequent components in operation deliver the antipsychotic agent in a pulsed or controlled manner over a period of up to twenty-four hours.

### FIELD OF THE INVENTION

The present invention relates to a novel formulation for the controlled delivery, over a period of time of up to twenty-four hours, of an antipsychotic agent selected from the group consisting of: a dibenzothiazepine derivative, for example, quetiapine or a salt thereof; lithium; and divalproex. The formulation is used, in particular, in the treatment of patients suffering from acute manic episodes associated with Bipolar I Disorder.

### BACKGROUND OF THE INVENTION

Quetiapine fumarate (2-[2-(4-dibenzo[b,f] [1.4]thiazepin-11-yl-1-piperazinyl)ethoxy]-ethanol fumarate (2:1) (salt)) is an antipsychotic agent belonging to a new chemical class, the dibenzothiazepine derivatives, and is marketed under the registered trademark SEROQUEL® by AstraZeneca Pharmaceuticals, LP, of Wilmington, Del. It is an antagonist at multiple neurotransmitter receptors in the brain: serotonin 5HT_{1A} and 5HT₂ receptors (IC₅₀ₛ=717 & 148 nM respectively), dopamine D₁ and D₂ receptors (IC₅₀ₛ=1268 & 329 nM respectively), histamine H₁ receptors (IC₅₀=30 nM), and adrenergic α₁ and α₂ receptors (IC₅₀ₛ=94 & 271 nM, respectively). Quetiapine fumarate activity is primarily due to the parent drug. The mechanism for its action, as with that of other antipsychotic agents, is unknown. However, it has been proposed that quetiapine's therapeutic activity in schizophrenia is mediated through a combination of dopamine type 2 (D₂) and serotonin type 2 (5HT₂) antagonism.

The multiple-dose pharmacokinetics of quetiapine are dose-proportional within the proposed clinical dose range, and quetiapine accumulation is predictable upon multiple dosing. Elimination of quetiapine is mainly by hepatic metabolism with a mean terminal half-life of about 6 hours within the proposed clinical dose range. Steady-state concentrations are expected to be achieved within two days of dosing.

Due to the high degree of bioavailability and rapid metabolism of the antipsychotic agent, the agent typically has to be administered multiple times. In addition, allowing the population of antipsychotic agent from one administration to clear from the patient's system prior to the administration of a subsequent population of antipsychotic agent has been thought to be a contributing factor in reducing or preventing patient tolerance.

Such multiple administrations, however, may result in problems related to patient compliance and increased heath care costs. Accordingly, it would be advantageous to provide a formulation which allows for the delivery of an antipsychotic agent in a controlled or delayed release profile. More specifically, it would be a tremendous benefit to patients suffering from acute manic episodes associated with Bipolar I Disorder if the agent could be formulated to be released in a two-phase or pulsatile manner so that the agent can provide its pharmacological activity over an extended period of time, in particular, a twenty-four hour period, instead of being rapidly metabolized. In this manner, patients suffering from acute manic episodes associated with Bipolar I Disorder would benefit from the therapeutic effects of the antipsychotic agent for extended periods of time without the need to take more than one dosage per day.

U.S. Pat. No. 4,879,288 to Warner et al., entitled “Novel Dibenzothiazepine Antipsychotic,” is incorporated by reference herein.

### SUMMARY OF THE INVENTION

An aspect of the present invention is a formulation comprising: (A) a first component which comprises a first population of an antipsychotic agent selected from the group consisting of: a dibenzothiazepine derivative; lithium; and divalproex; and (B) at least one subsequent component which comprises a subsequent population of an antipsychotic agent selected from the group consisting of: a dibenzothiazepine derivative; lithium; and divalproex; and which allows for the modified release of the agent.

Another aspect of the present invention is a dosage form comprising: (A) particles which comprise a first component which comprises a first population an antipsychotic agent selected from the group consisting of: a dibenzothiazepine derivative; lithium; and divalproex; and (B) particles which comprise a subsequent component which comprises a subsequent population of an antipsychotic agent selected from the group consisting of: a dibenzothiazepine derivative; lithium; and divalproex; and which allows for the modified release of the agent.

Another aspect of the present invention is a dosage form comprising: (A) mini-tablets which comprise a first component which comprises a first population an antipsychotic agent selected from the group consisting of: a dibenzothiazepine derivative; lithium; and divalproex; and (B) mini-tablets which comprise a subsequent component which comprises a subsequent population of an antipsychotic agent selected from the group consisting of: a dibenzothiazepine derivative; lithium; and divalproex; and which allows for the modified release of the agent.

Yet another aspect of the present invention is a method for the treatment of acute manic episodes associated with Bipolar I Disorder, comprising administering a therapeutically effective amount of a formulation which comprises (A) a first component which comprises a first population an antipsychotic agent selected from the group consisting of: a dibenzothiazepine derivative; lithium; and divalproex; and (B) at least one subsequent component which comprises a subsequent population of an antipsychotic agent selected from the group consisting of: a dibenzothiazepine derivative; lithium; and divalproex; and which allows for the modified release of the agent.

### DETAILED DESCRIPTION OF THE INVENTION

Formulations similar to those disclosed herein are disclosed and claimed in the U.S. Pat. Nos. 6,228,398 and 6,730,325 to Devane et al., both of which are incorporated by reference herein.

The present invention relates to a formulation that delivers an antipsychotic agent in a pulsed or bimodal manner. The antipsychotic agent is selected from the group consisting of: a dibenzothiazepine derivative, for example, quetiapine or a salt thereof (e.g., quetiapine fumarate); lithium; and divalproex. The formulation comprises: (A) a “first component” which comprises a first population of said antipsychotic agent; and (B) at least one “subsequent component” which comprises a subsequent population of said antipsychotic agent and which allows for the modified release of the agent. The agent included in each of these components may be the same or different from that included in the other component(s) and may be present in an amount that is the same or different from that present in the other component(s).

In an embodiment of the present invention, substantially all of one population of the antipsychotic agent is released prior to the release of the subsequent population of the antipsychotic agent. In instances when it is desirable to minimize patient tolerance by providing a dosage regime in which a population of the antipsychotic agent has cleared from a patient's system prior to the release of a subsequent population of the antipsychotic agent, the release of the subsequent population is delayed until such clearance occurs.

In an embodiment of the present invention, the release of a subsequent population of the antipsychotic agent is delayed for a period of at least two hours after administration of the formulation. The subsequent population is then released over the remainder of the twenty-four hours post-administration period.

In an embodiment of the present invention, the “first component” (as described above) allows for the release of the population of the antipsychotic agent contained therein substantially immediately after the administration of the formulation (hereafter, such a component is termed an “immediate release component”).

In another embodiment of the present invention, the “first component” (as described above) allows for the release of the population of the antipsychotic agent contained therein substantially immediately after a period of time has passed following the administration of the formulation (hereafter, such a component is termed a “delayed immediate release component”).

In an embodiment of the present invention, the subsequent component is a component which comprises a means which allows for the modified release of the population of the antipsychotic agent contained therein (hereafter, such a component is termed a “modified release component”). The release of this population of the antipsychotic agent is modified such that there is a period of time between the release of the previous population and the release of the present population (hereafter, this time will be known as the “lag time”). Such a modified release may be a delayed immediate release as described above or a controlled release (e.g., for up to twenty-four hours). The means which allows for the modified release may be a coating, a matrix, or both. The duration of the lag time may be varied by altering the formulation and/or the amount of the coating, the matrix, and/or other aspects of the components (e.g., the amount and nature of the antipsychotic agent or the amount and nature of inactive agents contained in the component). In one embodiment of the present invention, the lag time is four hours.

In an embodiment of the present invention, the release profile of the antipsychotic agent from the formulation mimics that of a desired plasma profile thereof. The profile may be one in which two or more pulses of high concentrations of the antipsychotic agent (peaks) are interspersed with periods of low concentration (troughs). Such a profile is called a “pulsatile profile”. A pulsatile profile with two peaks is referred to as being “bimodal”. A formulation which produces, upon administration, a pulsatile profile for the active agent therein is said to exhibit “pulsed release” of the agent.

A pulsatile profile may be achieved, for example, by the use of a formulation which comprises an immediate release component and a modified release component. The formulation may comprise additional modified release components as desired. A pulsatile release profile as produced from a single administration of the formulation of the present invention is advantageous when it is desired to deliver two or more pulses of the antipsychotic agent without the need for two or more administrations thereof. Depending on the duration of the lag time, if any, between the release of the various populations of the antipsychotic agent and the nature of the release (e.g., immediate, modified, etc.), the pulses in the plasma profile may be well separated and clearly defined (e.g., when the lag time is long) or they may be superimposed to a degree (e.g., when the lag time is short).

An example of the aforementioned means which allows for the modified release of the antipsychotic agent from a modified release component (hereafter, the “modified release means”) is a coating (hereafter, a “modified release coating”). Any coating material which modifies the release of the antipsychotic agent in the desired manner may be used. In particular, coating materials suitable for use in the practice of the invention include but are not limited to polymer coating materials, such as cellulose acetate phthalate, cellulose acetate trimaletate, hydroxy propyl methylcellulose phthalate, polyvinyl acetate phthalate, ammonio methacrylate copolymers such as those sold under the trademark Eudragit® RS and μL, poly acrylic acid and poly acrylate and methacrylate copolymers such as those sold under the trademark Eudragit® S and L, polyvinyl acetaldiethylamino acetate, hydroxypropyl methylcellulose acetate succinate, shellac; hydrogels and gel-forming materials, such as carboxyvinyl polymers, sodium alginate, sodium carmellose, calcium carmellose, sodium carboxymethyl starch, poly vinyl alcohol, hydroxyethyl cellulose, methyl cellulose, gelatin, starch, and cellulose based cross-linked polymers in which the degree of crosslinking is low so as to facilitate adsorption of water and expansion of the polymer matrix, hydroxypropyl cellulose, hydroxypropyl methylcellulose, polyvinylpyrrolidone, crosslinked starch, microcrystalline cellulose, chitin, aminoacryl-methacrylate copolymer (Eudragit® RS-PM, Rohm & Haas), pullulan, collagen, casein, agar, gum arabic, sodium carboxymethyl cellulose, (swellable hydrophilic polymers) poly(hydroxyalkyl methacrylate) (m. wt. .about. 5 k-5,000 k), polyvinylpyrrolidone (m. wt. about. 10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (m. wt. .about. 30 k-300 k), polysaccharides such as agar, acacia, karaya, tragacanth, algins and guar, polyacrylamides, Polyox® polyethylene oxides (m. wt. about. 100 k-5,000 k), AquaKeep™ acrylate polymers, diesters of polyglucan, crosslinked polyvinyl alcohol and poly N-vinyl-2-pyrrolidone, sodium starch glucolate (e.g., Explotab®; Edward Mandell C. Ltd.); hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides (e.g., Polyox®, Union Carbide), methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g., Eudragit®, Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof. As will be appreciated by the person skilled in the art, excipients such as plasticizers, lubricants, solvents and the like may be added to the coating. Suitable plasticizers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl mono glyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidized tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate. In an embodiment of the present invention, the coating material may be a material whose integrity is pH-dependent. In another embodiment of the present invention, the coating may comprise a mixture of methacrylate and ammonio methacrylate wherein the ratio of methacrylate to ammonio methacrylate is 1:1.

Another example of the aforementioned means is a matrix material (hereafter “modified release matrix material”). Any suitable modified release matrix material or suitable combination of modified release matrix materials may be used. Such materials are known to those skilled in the art. The term “modified release matrix material” as used herein includes hydrophilic polymers, hydrophobic polymers and mixtures thereof which are capable of modifying the release of an antipsychotic agent dispersed therein in vitro or in vivo. Modified release matrix materials suitable for the practice of the present invention include but are not limited to microcrystalline cellulose, sodium carboxymethylcellulose, hydroxyalkylcelluloses such as hydroxypropylmethylcellulose and hydroxypropylcellulose, polyethylene oxide, alkylcelluloses such as methylcellulose and ethylcellulose, polyethylene glycol, polyvinylpyrrolidone, cellulose acteate, cellulose acetate butyrate, cellulose acteate phthalate, cellulose acteate trimellitate, polyvinylacetate phthalate, polyalkylmethacrylates, polyvinyl acetate and mixtures thereof.

One of skill in the art would understand that certain of the above-described modified release means may be used also in the production of the above-described “delayed immediate release component”. In such an instance, the appropriate means should be one that it is capable of allowing for a substantially immediate release of the antipsychotic agent contained in the component after a period of time has passed following the administration of the formulation. For example, a delayed immediate release component may comprise a coating material whose integrity is pH-dependent. In such an embodiment, upon the arrival of the component in an environment which has a pH under which the integrity of the coating breaks down, the coating will break down and allow for the substantially immediate release of the antipsychotic agent contained in the component.

The modified release component may, for example, be in the form of an erodable formulation. In an erodable formulation, the formulation may employ a modified release coating and/or a modified release matrix material, either or both of which dissolve in water over time, thus losing their structural integrity. One manner in which this could occur would be that the active ingredient and modified release coating and/or matrix material dissolve after human ingestion over a controlled period of time.

The modified release component may also, for example, be in the form of a diffusion controlled formulation which allows for the gradual spread of the population of the antipsychotic agent in a liquid medium. An example of such a formulation is described in U.S. Pat. No. 6,586,006 to Roser et al., which is incorporated by reference herein.

The modified release component may also, for example, be in the form of an osmotic-controlled formulation. An example of such a formulation is described in U.S. Pat. No. 6,110,498 to Rudnic et al. The formulation described therein is one which dispenses a therapeutic agent having limited water solubility in solubilized form. The delivery system comprises a core that is free of swellable polymers and comprises nonswelling solubilizing agents and wicking agents. The solubilized therapeutic agent is delivered through a passageway in the semipermeable coating of the tablet. Another example of such a formulation is described in U.S. Pat. No. 6,814,979 to Rudnic et al. The formulation described therein comprises: (A) a semi-permeable wall that maintains its integrity during pharmaceutical delivery and which has at least one passage therethrough; and (B) a single, homogeneous composition within said wall, which composition consists essentially of (i) a pharmaceutically active agent, (ii) at least one non-swelling solubilizing agent which enhances the solubility of the pharmaceutically active agent, (iii) at least one non-swelling osmotic agent, and (iv) a non-swelling wicking agent dispersed throughout the composition which enhances the surface area contact of the pharmaceutical agent with the incoming aqueous fluid. Both of these patents are incorporated by reference herein.

In embodiments of the present invention in which quetiapine fumarate is used as an antipsychotic agent, it may be present either in the form of one substantially optically pure enantiomer or as a mixture, racemic or otherwise, of enantiomers. In an embodiment of the present invention, the quetiapine fumarate is present in each component of the formulation in an amount of from about 0.1 mg to about 1 g, preferably in an amount of from about 0.1 mg to 500 mg, more preferably in an amount of from 0.5 to 60 mg, and more preferably in an amount of from 2.5 to 30 mg.

In addition to the above, the formulation of the present invention may comprise also additional compounds, for example, an enhancer and a sensitizer. An enhancer is a compound which is capable of enhancing the absorption and/or bioavailability of an active agent. Examples of such an enhancer include: medium chain fatty acids and salts, esters, ethers and derivatives thereof (e.g., glycerides and triglycerides); non-ionic surfactants (e.g., non-ionic surfactants that can be prepared by reacting ethylene oxide with a fatty acid, a fatty alcohol, an alkylpehnol, or a sorbitan or glycerol fatty acid ester; cytochrome P450 inhibitors); and P-glycoprotein inhibitors and the like.

A formulation according to the present invention may be formulated into any suitable dosage form which facilitates the release of the antipsychotic agent. The dosage form may, for example, be a capsule (e.g., a hard or soft gelatin capsule) which contains, therein, the above described “first component” and one of more of the above described “subsequent component(s)”. The components may exist therein in various forms, for example, in the form of particles or mini-tablets. In one embodiment, a capsule comprises a particle comprising an immediate release component and/or a particle comprising a delayed immediate release component and a particle comprising a modified release component. The particle comprising the modified release component and the particle comprising the delayed immediate release component may, for example, be particles comprising the antipsychotic drug and one or more modified release means. For example, the particle may be made out of a modified release matrix material and/or coated with a modified release coating. In the case of a delayed immediate release particle, the modified release means may, for example, be a coating whose integrity is pH-dependent, as described above. The particle comprising the immediate release component may, for example, be in the form of a particle which comprises the antipsychotic drug but not a modified release means. In embodiments in which the capsule comprises mini-tablets, the capsule may, for example, comprise a mini-tablet comprising an immediate release component and/or a mini-tablet comprising a delayed immediate release component and a mini-tablet comprising a modified release component. The mini-tablets may be formed, for example, by compressing the above-described particles (e.g., a modified release mini-tablet may be formed by compressing modified release particles). In addition to the above, the dosage form may be, for example, in the form of a multilayer tablet with one layer comprising an immediate release component or a delayed immediate release component and another layer comprising a modified release component. Additional examples of dosage forms include rapidly dissolving dosage forms such as an effervescent dosage form or a fast-melt dosage form.

The present invention further provides a method of treating a patient suffering from acute manic episodes associated with bipolar disorder utilizing the formulation of the present invention.

It will be apparent to those skilled in the art that various modifications and variations can be made in the methods and formulations of the present invention without departing from the spirit or scope of the invention. Thus, it is intended that the present invention cover the modifications and variations of this invention, provided they come within the scope of the appended claims and their equivalents.

We claim:

1. A formulation comprising: (A) a first component which comprises a first population of an antipsychotic agent selected from the group consisting of: a dibenzothiazepine derivative; lithium; and divalproex; and (B) at least one subsequent component which is a modified release component and which comprises a subsequent population of an antipsychotic agent selected from the group consisting of: a dibenzothiazepine derivative; lithium; and divalproex.

2. A formulation according to claim 1 wherein said formulation comprises only one subsequent component.

3. A formulation according to claim 1 wherein said modified release component comprises a modified release means.

4. A formulation according to claim 3 wherein said modified release means is a modified release coating or a modified release matrix material.

5. A formulation according to claim 1 wherein said antipsychotic agent is a dibenzothiazepine derivative.

6. A formulation according to claim 1 wherein said antipsychotic agent is quetiapine or a salt thereof.

7. A formulation according to claim 1 wherein said antipsychotic agent is quetiapine fumarate.

8. A formulation according to claim 1 wherein said subsequent component releases said subsequent population over a period of up to twenty-four hours.

9. A formulation according to claim 1 wherein said subsequent component comprises an erodable formulation.

10. A formulation according to claim 1 wherein said subsequent component comprises a diffusion controlled formulation.

11. A formulation according to claim 1 wherein said subsequent component comprises an osmotic controlled formulation.

12. A formulation according to claim 7 wherein quetiapine fumarate is present in each component in an amount of from about 0.1 mg to about 1 g.

13. A formulation according to claim 1 wherein said first population is substantially released prior to the release of a subsequent population.

14. A formulation according to claim 3 wherein said modified release means is a modified release coating.

15. A formulation according to claim 14 wherein the integrity of said coating is pH-dependent.

16. A formulation according to claim 14 wherein said coating comprises methacrylate copolymers.

17. A formulation according to claim 14 wherein said coating comprises a mixture of methacrylate and ammonio methacrylate copolymers in a ratio sufficient to achieve a pulse of the active ingredient following a time delay.

18. A formulation according to claim 17 wherein said ratio of methacrylate to ammonio methacrylate copolymers is 1:1.

19. A dosage form comprising the formulation of claim 1 wherein said dosage form comprises: (A) particles which comprise said first component; and (B) particles which comprise said subsequent component.

20. A dosage form according to claim 14 wherein said particles are contained in a capsule.

21. A dosage form comprising the formulation of claim 1 wherein said dosage form comprises: (A) mini-tablets which comprise said first component; and (B) mini-tablets which comprise said subsequent component.

22. A method for the treatment of acute manic episodes associated with Bipolar I Disorder in a patient, comprising administering to said patient a therapeutically effective amount of a formulation according to claim 1.
